(19) United States
(12) Plant Patent Application Publication
(10) Pub. No.: US 2012/0222184 P1
(43) Pub. Date: Aug. 30, 2012

(54) Heuchera plant named 'Root Beer'

(50) ;

(75) Inventor: Janet N. Egger, Wilsonville, OR (US)

(73) Assignee: Terra Nova Nurseries, Inc.

(21) Appl. No.: 12/932,453

(22) Filed: Feb. 25, 2011

(51) Int. Cl. A01H 5/00

(52) U.S. Cl. PLT/440

(57) ABSTRACT

A new and distinct Heuchera plant characterized by medium red brown leaves year round, very free flowering on medium to short flower stalks, creamy yellow flowers on red peduncles, well branched, mounding habit, and excellent vigor.

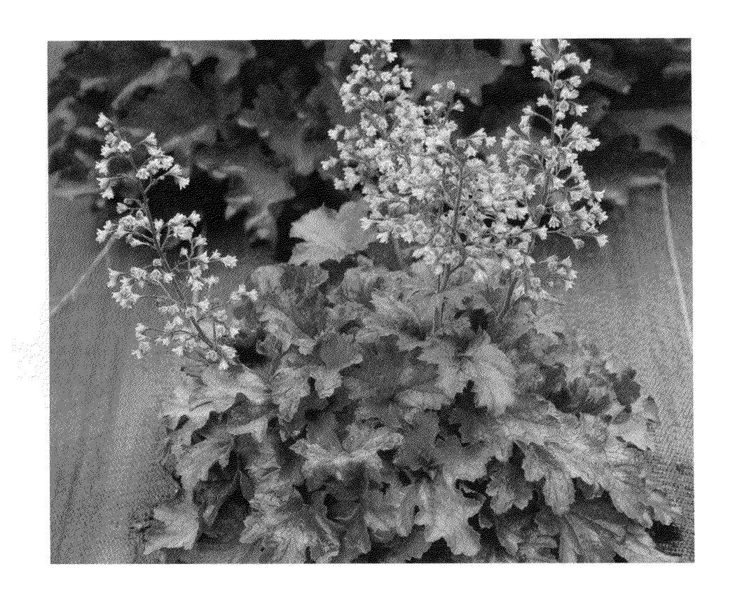

### BOTANICAL DENOMINATION

Heuchera sp.

### VARIETY DESIGNATION

‘Root Beer’

### CROSS REFERENCE TO

Heuchera ‘Cherry Cola’ (U.S. Plant patent pending)

### BACKGROUND OF THE INVENTION

The present invention relates to a new and distinct cultivar of Heuchera and given the cultivar name of ‘Root Beer’. Heuchera is in the family Saxifragaceae. Heuchera ‘Root Beer’ originated from a cross between Heuchera 747-5, a proprietary non-commercial hybrid, as the seed parent, and Heuchera 766-5, a proprietary non-commercial hybrid, as, as the pollen parent.

Compared to the seed parent, Heuchera 747-5, the new cultivar has smaller leaf size and red brown rather than rose to olive leaf color.

Compared to the pollen parent, Heuchera 766-5, the new cultivar has creamy yellow rather than light pink to white flowers and red brown rather than purple black leaves.

Compared to its sibling Heuchera ‘Cherry Cola’, the new cultivar has darker colored leaves and creamy yellow rather red than flowers.

This new Heuchera is uniquely distinguished by:
1. medium red brown leaves year round,
2. very free flowering on medium to short flower stalks,
3. creamy yellow flowers on red peduncles,
4. well branched, mounding habit, and
5. excellent vigor.

This new cultivar has been reproduced only by asexual propagation (tip cuttings and tissue culture). Each of the progeny exhibits identical characteristics to the original plant. Asexual propagation by cuttings and tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may change with variations in environment without a change in the genotype of the plant.

### BRIEF DESCRIPTION OF THE DRAWING

The photograph shows a 1-year-old plant of Heuchera ‘Root Beer’ growing in the ground in full sun in the trial field in August in Canby, Oreg.

### DETAILED PLANT DESCRIPTION

The following is a detailed description of the new Heuchera cultivar based on observations taken in April of 2-year-old specimens growing in the ground in the garden in full sun in Canby, Oreg. Canby is Zone 8 on the USDA Hardiness map. Temperatures range from a high of 95 degrees F. in August to an average of 32 degrees F. in January. Normal rainfall in Canby is 42.8 inches per year. The color descriptions are all based on The Royal Horticultural Society Colour Chart, 5^{th} edition.
Plant:
   Form.—basal rosette, herbaceous perennial.
   Hardiness.—USDA Zone 4 to 9.
   Size.—15 cm tall from top of the foliage to the ground and 35 cm wide.
   Habit .—mounding.
   Vigor .—excellent.
Leaf:
   Type.—simple.
   Arrangement.—rosette.
   Shape.—broadly ovate to orbicular.
   Lobing/Division.—5 to 7 shallow lobes, shallow secondary lobes.
   Venation.—palmate.
   Margins.—broadly crenate.
   Apex.—mucronulate.
   Base.—cordate, lobes usually overlapping at the base.
   Blade size.—grows to 8.5 cm long and 9.3 cm wide.
   Texture.—leathery.
   Surface texture.—pubescent on top side, glandular on bottom.
   Petiole description.—grows to 15 cm long and 2.5 mm wide, glandular hairs, Red Purple 59B.
   Leaf color.—topside, spring leaves are Greyed Purple 185A, bottom side Red Purple 59B; summer leaves are Greyed Purple N186C with slight white veiling, White N155B; winter leaves topside are Greyed Purple 183A with tints of Brown 200C, bottom side Red Purple 59B.
Inflorescence:
   Size.—grows to 7 cm wide and 31 cm long.
   Type.—thyrse.
   Number of flowers per thyrse.—about 70.
   Number of thyrse.—about 20 in the first spring flush.
   Peduncle.—grows to 30 cm long, 4 mm wide at base, Greyed Purple 187B, glandular hairs, with 1 to 3 pedicellate cauline leaves (grow to 6 cm long and 6 mm wide, ovate to orbicular, 3 to 5 lobed, margins crenate, tip mucronulate, base cordate, glandular, topside Greyed Red 181 B, bottom side Greyed Purple 185A).
   Pedicel.—variable in size, with glandular hairs, Greyed Purple 187B.
   Bloom period.—April through June in Canby, Oreg.
Flower bud:
   Size.—2.5 mm wide and 4 mm long.
   Description.—glandular puberulent, ovoid, down facing.
   Color.—bottom half Greyed Purple 185B, top half Greyed Yellow 161 B tinted Greyed Purple 185B.
Flower:
   Type.—perfect.
   Shape.—campanulate.
   Size.—6 mm long and 4 mm wide.
   Corolla description.—5 petals, 3 mm long and 1 mm wide, spatulate with 0 to 2 jagged side lobes, margins entire, tip acute, sparsely glandular on both sides, White NN155C.
   Calyx description.—campanulate, 5 mm long and 4 mm wide, with 5 lobes divided ½ way to the base, each 1.7 mm wide and 2.5 mm long, glandular hairs outside and inside, tips obtuse, margin entire; color outside on bottom half Greyed Yellow 161A and on top half Greyed Yellow 161B with tips tinted Greyed Purple 185B, inside color Greyed Yellow 161C.
   Stamen description.—5 in number, 1 mm long, no pollen, male sterile.
   Pistil description.—1, 2-beaked, 2 mm long and 1.5 mm wide; Greyed Yellow 160C overall.
   Fragrance.—none.
   Lastingness.—each thyrse blooms for about 3 weeks on the plant.
Fruit:
   Type.—two-beaked ovoid capsule.
   Fertility.—low.
   Color.—Greyed Brown 199C.
Seed:
   Shape.—linear.
   Size.—2 mm long.
   Color.—Black 202A.
Pest and disease tolerance: Excellent disease resistance to powdery mildew, the common problem of Heuchera. Susceptible to Heuchera rust and root weevils.

I claim:

1. A new and distinct Heuchera plant as herein illustrated and described.
